(19) United States
(12) Patent Application Publication
(10) Pub. No.: US 2021/0366051 A1
(43) Pub. Date: Nov. 25, 2021

(54) COMPUTER SYSTEM AND LINKAGE CONTROL METHOD

(71) Applicant: Hitachi, Ltd., Tokyo (JP)

(72) Inventors: Jiyun YOSHIHARA, Tokyo (JP); Kozo KATO, Tokyo (JP); Shinichiro SAITO, Tokyo (JP); Tomoya KUGA, Tokyo (JP)

(21) Appl. No.: 17/269,358

(22) PCT Filed: Aug. 25, 2020
(86) PCT No.: PCT/JP2020/032089

§ 371 Date: Feb. 18, 2021

(30) Foreign Application Priority Data
Oct. 9, 2019 (JP) 2019-185884

(51) Int. Cl. G06Q 40/08; G06Q 10/06

(52) U.S. Cl. CPC G06Q 40/08; G06Q 10/0637; G06Q 10/0631

(57) ABSTRACT

In a computer system for performing linkage control of information between organizations, scenario management information for managing a scenario in which trigger information related to an event related to a user is associated with a target organization as a target of the linkage control accompanying an occurrence of the event is held, and when receiving information from an arbitrary organization, an arithmetic device searches for the scenario including the received information as the trigger information, identifies the target organization based on the scenario and the received information, and transmits shared information generated from the received information to the target organization.

### INCORPORATION BY REFERENCE

This application claims priority of Japanese Patent Application No. 2019-185884, filed on Oct. 9, 2019, and is incorporated herein by reference.

### TECHNICAL FIELD

The present invention relates to a system and a method for realizing linkage of information between organizations.

### BACKGROUND ART

In order to realize the Society 5.0 and the SDGs (Sustainable Development Goals), efforts are currently being accelerated to create new services and industries by linking and utilizing data scattered in private companies, the national government, local governments, and the like across organizations.

Regarding the above problem, Patent Literature 1 is disclosed in terms of a system for generally authenticating an individual to be associated with personal information.

Patent Literature 1 describes “A general system enabling personal authentication in an organization is provided. The system consists of an online data processing system such as a WEB server accessed from a user terminal and an authentication organization system connected through a network. The online data processing system includes electronic certificate reception means for receiving an electronic certificate transmitted from a user terminal and authentication means for performing user authentication by transmitting and receiving data to/from the authentication organization system. The authentication organization system is provided with an authentication database, and stores business type identification data such as a company code and personal authentication data for authenticating an individual in an organization by associating the business type with the individual. User authentication is performed based on authentication information including at least authority information of an individual in an organization extracted from the authentication database.”

### CITATION LIST

### Patent Literature

[Patent Literature 1] International Publication 2003/105002

### SUMMARY OF INVENTION

### Technical Problem

The prior art provides a repository for securely sharing information between organizations. However, when data is acquired from the repository, the organization needs to perform an explicit operation. The prior art does not provide a technique for propagating information between organizations to realize linkage of services.

An object of the present invention is to provide a system and a method for realizing linkage of information between organizations without performing an explicit operation.

### Solution to Problem

A representative example of the invention disclosed in the application is as follows. That is, provided is a computer system for performing linkage control of information between organizations, the system comprising at least one computer having an arithmetic device and a storage device connected to the arithmetic device, wherein scenario management information for managing a scenario in which trigger information related to an event related to a user is associated with a target organization as a target of the linkage control accompanying an occurrence of the event is held, and wherein when receiving information from an arbitrary organization, the arithmetic device refers to the scenario management information to search for the scenario including the received information as the trigger information, identifies the target organization based on the scenario and the received information, and transmits shared information generated from the received information to the target organization.

### Advantageous Effects of Invention

According to the present invention, it is possible to realize linkage of information between organizations without performing an explicit operation. Problems, configurations, and effects other than those described above will be clarified by the following description of the embodiments.

### BRIEF DESCRIPTION OF DRAWINGS

FIG. 1 is a diagram for describing the concept of a linkage infrastructure system of a first embodiment.

FIG. 2 is a diagram for showing a configuration example of the linkage infrastructure system of the first embodiment.

FIG. 3 is a diagram for describing an example of a data structure of organization management information of the first embodiment.

FIG. 4 is a diagram for describing an example of a data structure of scenario template management information of the first embodiment.

FIG. 5 is a diagram for describing an example of a data structure of scenario registration control information of the first embodiment.

FIG. 6 is a diagram for describing an example of a data structure of personal scenario management information of the first embodiment.

FIG. 7 is a diagram for describing an example of a data structure of implementation scenario management information of the first embodiment.

FIG. 8 is a flowchart for describing an example of a personal scenario registration process executed by the linkage infrastructure system of the first embodiment.

FIG. 9 is a diagram for showing an example of a registration screen presented by the linkage infrastructure system of the first embodiment.

FIG. 10 is a flowchart for describing an example of a linkage control process executed by the linkage infrastructure system of the first embodiment.

FIG. 11 is a diagram for showing a configuration example of a linkage infrastructure system of a second embodiment.

FIG. 12 is a diagram for showing a concrete example of scenario template management information of a third embodiment.

FIG. 13 is a diagram for showing a concrete example of personal scenario management information of the third embodiment.

### DESCRIPTION OF EMBODIMENTS

Hereinafter, embodiments of the present invention will be described using the drawings. However, the present invention is not interpreted by being limited to the description contents of the embodiments shown below. It will be easily understood by those skilled in the art that concrete configurations can be changed without departing from the idea or spirit of the present invention.

In the configuration of the invention described below, the same or similar configurations or functions are followed by the same signs, and duplicate explanations are omitted.

The notations of “first”, “second”, “third”, and the like in the specification and the like are added to identify the constitutional elements, and do not necessarily limit numbers or orders.

### First Embodiment

In a first embodiment, a basic configuration of the present invention will be described. Concrete use cases will be described using a third embodiment.

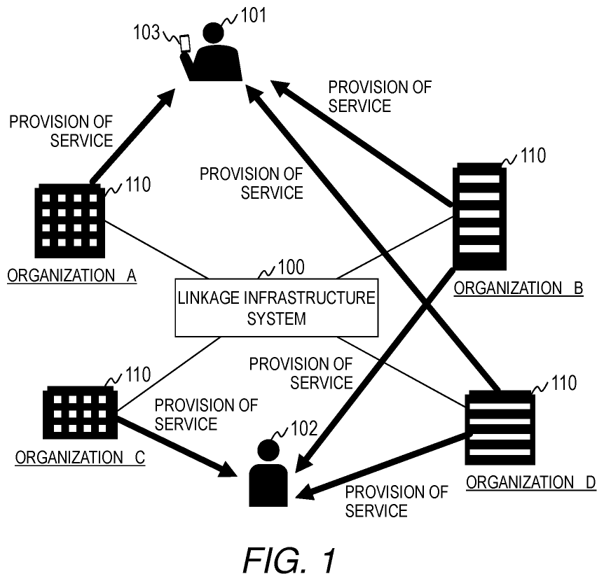

FIG. 1 is a diagram for describing the concept of a linkage infrastructure system 100 of the first embodiment.

Organizations 110 provide services to a user 101 or a person involved 102 of the user 101. The organizations 110 are, for example, public offices providing administrative services, hospitals providing medical services, insurance companies providing insurance services, and the like. Systems operated by the organizations 110 may be either on-premise systems or cloud systems. The systems include an application server, a database server, an authentication information generation server, and the like. Information about the user 101 is stored in the database server.

The linkage infrastructure system 100 is connected to the systems operated by the plural organizations 110 through networks such as the Internet, an intranet, a WAN (Wide Area Network), and a LAN (Local Area Network). Note that the connection method of the networks may be either wired or wireless. In response to an event occurring to the user 101, the linkage infrastructure system 100 realizes the linkage of information between the organizations 110 related to the event.

The user 101 uses a user terminal 103 such as a personal computer or a smart phone to register setting information necessary for the linkage of information between the organizations 110 into the linkage infrastructure system 100.

The organization 110 generates a private key and a public key by using the system, and transmits the public key to the linkage infrastructure system 100. In addition, the user 101 generates a private key and a public key by using the user terminal 103, and transmits the public key to the linkage infrastructure system 100.

In addition, when information related to the user 101 is generated, the organization 110 is set to notify the linkage infrastructure system 100 of the information by using the system. Note that the information to be generated includes identification information of the user 101.

The linkage infrastructure system 100 can provide various services through the organizations 110 to the user 101 and the person involved 102 by realizing the sharing of information between the organizations 110 in response to the event that occurred.

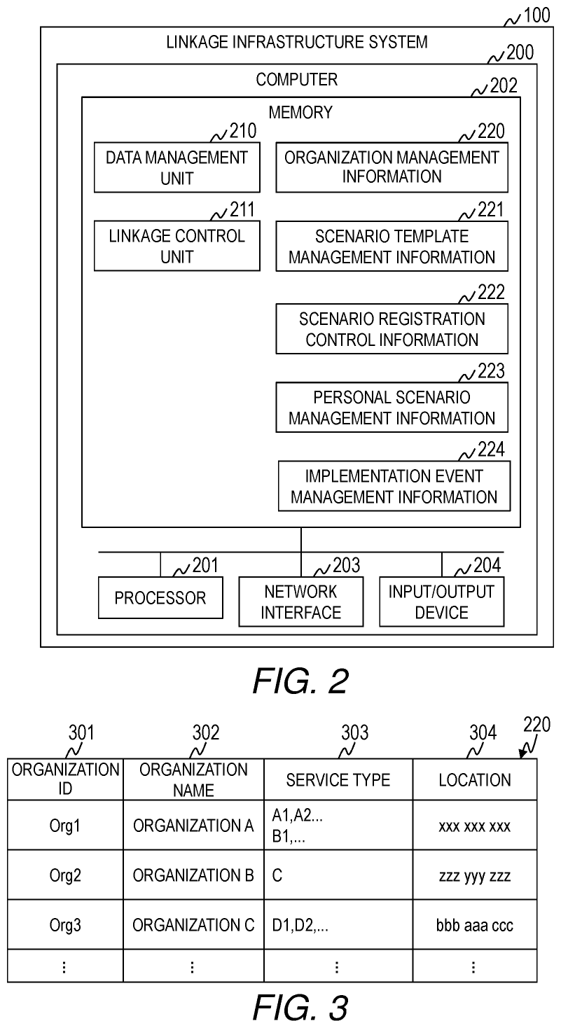

FIG. 2 is a diagram for showing a configuration example of the linkage infrastructure system 100 of the first embodiment.

The linkage infrastructure system 100 includes at least one computer 200. The computer 200 has a processor 201, a memory 202, a network interface 203, and an input/output device 204. The units of hardware are connected to each other through an internal bus or the like.

The network interface 203 is an interface for communicating with an external device through a network. The input/output device 204 is a device for inputting data and outputting data. The input/output device 204 includes, for example, a keyboard, a mouse, a touch panel, a display, a printer, and the like.

The processor 201 is an arithmetic device for executing various processes. The processor 201 executes a process in accordance with a program stored in the memory 202, thereby operating as a functional unit (module) for realizing a specific function. In the following description, when a process is described using the functional unit as the subject, it indicates that the processor 201 executes a program for realizing the functional unit.

The memory 202 is a storage device for storing programs and information. The memory 202 includes a work area temporarily used by the program. The memory 202 of the first embodiment stores programs for realizing a data management unit 210 and a linkage control unit 211, and also stores organization management information 220, scenario template management information 221, scenario registration control information 222, personal scenario management information 223, and implementation scenario management information 224. Note that information for managing the public keys transmitted from the user 101 and the organization 110 is stored in the memory 202, but is omitted.

The organization management information 220 is information for managing the organizations 110 that share information through the linkage infrastructure system 100. The data structure of the organization management information 220 will be described in detail by using FIG. 3.

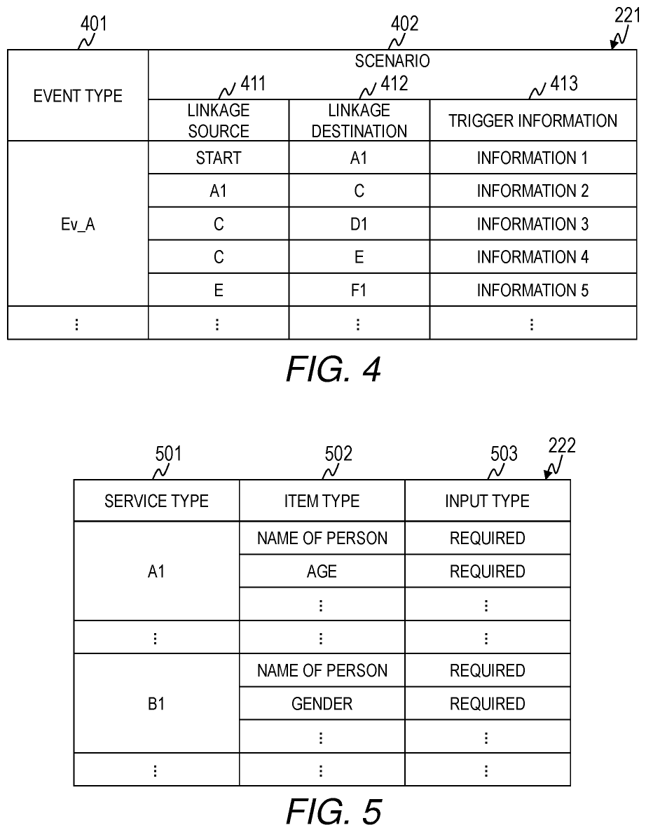

The scenario template management information 221 is information for managing a template of a scenario showing a flow of the linkage of information accompanying the occurrence of the event of the user 101. The data structure of the scenario template management information 221 will be described in detail by using FIG. 4.

The scenario registration control information 222 is information for managing items of information necessary for the linkage of information between the organizations 110. The data structure of the scenario registration control information 222 will be described in detail by using FIG. 5. Note that the information necessary for the linkage is personal information and the like. In addition, the items of the personal information are name, age, sex, occupation, address, phone number, driver's license number, insurer number, and the like.

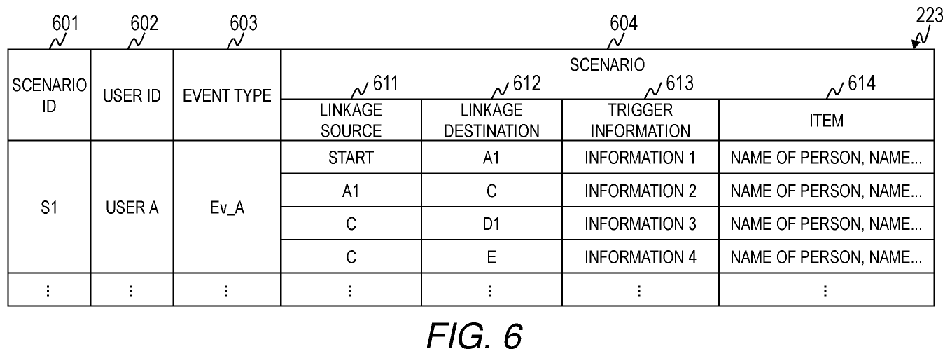

The personal scenario management information 223 is information for managing a personal scenario that is a scenario set by the user 101. The data structure of the personal scenario management information 223 will be described in detail by using FIG. 6.

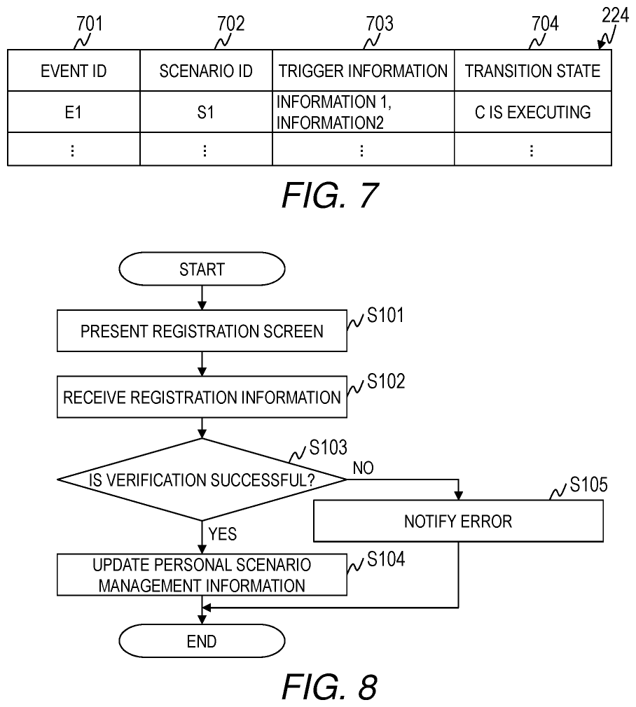

The implementation scenario management information 224 is information for managing the state of the personal scenario that is currently being implemented. The data structure of the implementation scenario management information 224 will be described in detail by using FIG. 7.

The data management unit 210 registers, deletes, and updates data. For example, the data management unit 210 registers the personal scenario based on an input from the user 101. The linkage control unit 211 performs linkage control of information based on the personal scenario.

Note that regarding the functional units provided in the computer 200, plural functional units may be combined into one functional unit, or one functional unit may be divided into plural functional units for each function.

Note that the computer 200 may include a storage device such as an HDD (Hard Disk Drive) or an SSD (Solid State Drive). The programs and information stored in the memory 202 may be stored in a storage device. In this case, the processor 201 reads the programs and information from the storage device, and loads the same into the memory 202.

FIG. 3 is a diagram for describing an example of the data structure of the organization management information 220 of the first embodiment.

The organization management information 220 stores an entry consisting of an organization ID 301, an organization name 302, a service type 303, and a location 304. There is one entry for one organization 110.

The organization ID 301 is a field that stores identification information for uniquely identifying an entry of the organization management information 220.

The organization name 302 is a field that stores the name of the organization 110. The organization name 302 stores, for example, a company name, and a public office name, and the like.

The service type 303 is a field that stores a value indicating the type of service provided by the organization 110. The service type 303 stores values corresponding to the number of services provided by the organization 110.

The location 304 is a field that stores the address of the home or the like of the organization 110.

Note that the entry may include fields other than those described above. For example, the entry may include a field for storing a URL or the like for accessing the system operated by the organization 110. In addition, it is not necessary for the entry to include all the fields described above.

FIG. 4 is a diagram for describing an example of the data structure of the scenario template management information 221 of the first embodiment.

The scenario template management information 221 stores an entry consisting of an event type 401 and a scenario 402. There is one entry for one scenario.

The event type 401 is a field that stores a value indicating the type of event. It is assumed that the type of event is set in advance.

The scenario 402 is a field group that stores a template of the scenario corresponding to the event type 401. The scenario 402 includes a linkage source 411, a linkage destination 412, and trigger information 413. One entry includes one or more rows consisting of the linkage source 411, the linkage destination 412, and the trigger information 413.

The linkage source 411 is a field that stores a value indicating the type of service provided by the organization 110 of the linkage source. The linkage destination 412 is a field that stores a value indicating the type of service provided by the organization 110 of the linkage destination. The trigger information 413 is a field that stores a value indicating the type of information (trigger information) serving as an opportunity to start the linkage of information from the organization 110 of the linkage source to the organization 110 of the linkage destination.

It is assumed that the scenario template management information 221 is set in advance by the administrator or the like of the linkage infrastructure system 100. Note that the scenario template management information 221 can be updated at an arbitrary timing.

Note that plural scenarios may be defined for the events of the same type. Note that the entry may include fields other than those described above. For example, a field that stores a condition for starting the linkage may be provided in the scenario 402. In addition, it is not necessary for the entry to include all the fields described above.

Note that when the organization 110 of the linkage destination is uniquely determined for the trigger information, it is not necessary for the entry to include the linkage source 411.

Note that although the entries on an event basis are described in order to easily understand the present invention, the present invention is not limited to thereto. The entry stored in the scenario template management information 221 may consist of the event type 401, the linkage source 411, the linkage destination 412, and the trigger information 413.

FIG. 5 is a diagram for describing an example of the data structure of the scenario registration control information 222 of the first embodiment.

The scenario registration control information 222 stores an entry consisting of a service type 501, an item type 502, and an input type 503. There is one entry for one type of service. One entry includes one or more rows consisting of the item type 502 and the input type 503.

The service type 501 is a field that stores a value indicating the type of service. The service type 501 stores a value indicating one type of service.

The item type 502 is a field that stores an item included in the information transmitted to the organization 110 providing the service corresponding to the service type 501.

The input type 503 is a field that stores a value indicating whether or not input of an item is required. The input type 503 stores either “required” indicating that the input is required or “option” indicating that the input is not required.

Note that the entry may include fields other than those described above. In addition, it is not necessary for the entry to include all the fields described above.

FIG. 6 is a diagram for describing an example of the data structure of the personal scenario management information 223 of the first embodiment.

An entry consisting of a scenario ID 601, a user ID 602, an event type 603, and a scenario 604 is stored. There is one entry for a personal scenario.

The scenario ID 601 is a field that stores identification information of a personal scenario. The user ID 602 is a field that stores identification information of the user 101. The event type 603 is a field that stores a value indicating the type of event.

The scenario 604 is a field group that stores the personal scenario. The scenario 604 includes a linkage source 611, a linkage destination 612, trigger information 613, and an item 614. One entry includes one or more rows consisting of the linkage source 611, the linkage destination 612, the trigger information 613, and the item 614. In the following description, the row is also described as linkage information.

The linkage source 611 is a field that stores a value indicating the type of service provided by the organization 110 of the linkage source. The linkage destination 612 is a field that stores a value indicating the type of service provided by the organization 110 of the linkage destination. The trigger information 613 is a field that stores a value indicating the type of trigger information. The item 614 is a field that stores a value indicating an item of information to be transmitted to the organization 110 of the linkage destination.

Note that the entry may include fields other than those described above. In addition, it is not necessary for the entry to include all the fields described above.

FIG. 7 is a diagram for describing an example of the data structure of the implementation scenario management information 224 of the first embodiment.

The implementation scenario management information 224 stores an entry consisting of an event ID 701, a scenario ID 702, trigger information 703, and a transition state 704.

The event ID 701 is a field that stores identification information of an event. The scenario ID 702 is a field that stores identification information of a personal scenario being executed with the occurrence of the event. The trigger information 703 is a field that stories a value indicating the type of trigger information received so far. The transition state 704 is a field that stores a value indicating the progress status of the scenario.

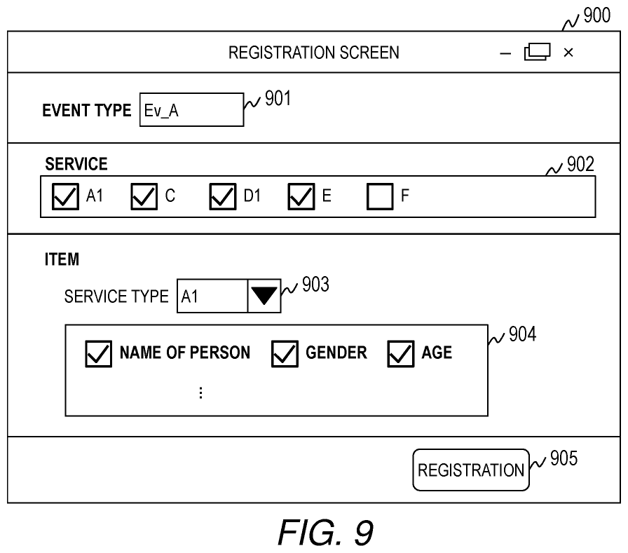

FIG. 8 is a flowchart for describing an example of a personal scenario registration process executed by the linkage infrastructure system 100 of the first embodiment. FIG. 9 is a diagram for showing an example of a registration screen presented by the linkage infrastructure system 100 of the first embodiment.

The user 101 transmits a registration request for a personal scenario to the linkage infrastructure system 100 by using the user terminal 103.

When receiving the registration request, the linkage infrastructure system 100 calls the data management unit 210 to start the personal scenario registration process.

First, the data management unit 210 presents a registration screen 900 to the user 101 (Step S101).

Here, a configuration of the registration screen 900 will be described. The registration screen 900 includes an event type input field 901, a service display field 902, a service selection field 903, an item selection field 904, and a registration button 905.

The event type input field 901 is a field for selecting the type of event. The service display field 902 is a field for displaying services available when an event occurs. The service display field 902 displays the types of services and check boxes for selecting services to be used.

The service selection field 903 is a field for selecting a service for setting an item. The service selection field 903 displays a list of the services selected in the service display field 902 as a pull-down menu.

The item selection field 904 is a field for selecting an item included in the information transmitted to the organization 110 providing the service corresponding to the service selection field 903. The item selection field 904 displays the types of items and check boxes for selecting items.

The registration button 905 is an operation button for transmitting the registration information set through the registration screen 900.

The above is the description of the registration screen 900. Next, a control method of the registration screen 900 will be described.

The data management unit 210 presents the registration screen 900 on which the event type input field 901, the service display field 902, the service selection field 903, and the item selection field 904 are blank.

(S101-1) When the user 101 sets a value in the event type input field 901, the data management unit 210 refers to the scenario template management information 221 to search for an entry in which the value of the event type 401 matches the value input in the event type input field 901. The data management unit 210 identifies available services based on the scenario 402 of the searched entry.

At this time, the user terminal 103 generates empty registration information, and adds the identification information of the user and the type of event to the registration information.

(S101-2) The data management unit 210 displays the types and check boxes of the identified services in the service display field 902. The data management unit 210 stores the types of services checked by the user.

Note that information cannot be linked depending on a combination of services. Accordingly, the data management unit 210 determines whether or not information can be linked by the combination of services selected by the user based on the scenario 402 of the searched entry. If information cannot be linked by the combination of services selected by the user, the data management unit 210 notifies an alert through the registration screen 900.

(S101-3) The data management unit 210 displays a list of the services selected by the user in the service selection field 903 as a pull-down menu.

(S101-4) When the type of service is set in the service selection field 903, the data management unit 210 refers to the scenario registration control information 222 to search for an entry in which the value of the service type 501 matches the value of the service selection field 903. The data management unit 210 displays items and check boxes in the item selection field 904 based on the item type 502 of the searched entry. The data management unit 210 may control not to accept a user operation by automatically checking the check box of the item for which the input type 503 is “required”.

At this time, the user terminal 103 adds data obtained by associating the operation contents of the item selection field 904 with the value of the service selection field 903 to the registration information.

(S101-5) When setting of the items for all the services selected by the user are completed, the data management unit 210 activates the registration button 905.

Note that when the registration button 905 is operated, the user terminal 103 generates an electronic signature, and transmits the registration information with the electronic signature to the linkage infrastructure system 100.

The above is the description of the control method of the registration screen 900. FIG. 8 will be described again.

When receiving the registration information through the registration screen 900 (Step S102), the data management unit 210 verifies the electronic signature included in the registration information by using the public key of the user, and determines whether or not the verification has been successful (Step S103).

If the verification has been successful, the data management unit 210 updates the personal scenario management information 223 (Step S104). Thereafter, the data management unit 210 terminates the personal scenario registration process. Specifically, the following processes are executed.

(S104-1) The data management unit 210 adds an entry to the personal scenario management information 223, and sets identification information to the scenario ID 601 of the added entry. In addition, the data management unit 210 sets the identification information of the user included in the registration information to the user ID 602 of the added entry, and sets the type of event included in the registration information to the event type 603.

(S104-2) The data management unit 210 refers to the scenario template management information 221 to search for an entry in which the value of the event type 401 matches the type of event included in the registration information.

(S104-2) The data management unit 210 sets a row (linkage information) to the scenario 604 based on the searched entry and the type and item of the service included in the registration information. The above is the description of the process of Step S104.

If the verification has failed, the data management unit 210 notifies the user terminal 103 of an error (Step S105). Thereafter, the data management unit 210 terminates the personal scenario registration process.

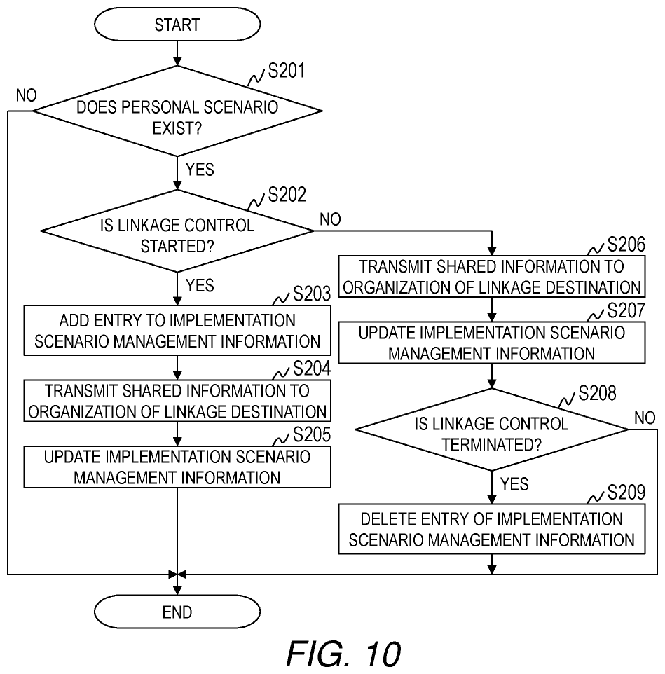

FIG. 10 is a flowchart for describing an example of a linkage control process executed by the linkage infrastructure system 100 of the first embodiment.

When receiving information from the organization 110, the linkage infrastructure system 100 calls the linkage control unit 211 to start the linkage control process. Note that the information transmitted by the organization 110 includes an electronic signature. In the following description, it is assumed that the verification of the electronic signature is successful.

The linkage control unit 211 determines whether or not a personal scenario using the received information as trigger information exists (Step S201). Specifically, the following processes are executed.

(S201-1) The linkage control unit 211 refers to the personal scenario management information 223 to determine whether or not an entry in which the identification information of the user included in the trigger information is set to the user ID 602 exists. If there is no entry satisfying the above-described condition, the linkage control unit 211 determines that no personal scenario exists.

(S201-2) If an entry in which the identification information of the user included in the trigger information is set to the user ID 602 exists, the linkage control unit 211 determines whether or not an entry including a row (linkage information) in which the identification information of the received trigger information is set to the trigger information 613 exists in the searched entry. If an entry satisfying the above-described condition exists, the linkage control unit 211 determines that a personal scenario exists. In this case, the linkage control unit 211 acquires the identification information of the personal scenario from the scenario ID 601 of the searched entry. The above is the description of the process of Step S202.

If it is determined that there is no personal scenario, the linkage control unit 211 terminates the linkage control process.

If it is determined that the personal scenario exists, the linkage control unit 211 determines whether or not the linkage control is started (Step S202).

Specifically, the linkage control unit 211 refers to the entry of the personal scenario identified in Step S201, and determines whether or not the type of trigger information set to the trigger information 613 of the row in which the linking source 611 is “start” matches the type of the received trigger information. If the above-described condition is satisfied, the linkage control unit 211 determines that the linkage control is started.

If it is determined that the linkage control is started, the linkage control unit 211 adds an entry to the implementation scenario management information 224 (Step S203).

Specifically, the linkage control unit 211 adds an entry to the implementation scenario management information 224, and sets the identification information of the event to the event ID 701 of the added entry. The linkage control unit 211 sets the value of the scenario ID 601 of the entry of the personal scenario identified in Step S201 to the scenario ID 702 of the added entry. The linkage control unit 211 stores a value indicating the type of the received trigger information in the trigger information 703 of the added entry.

Next, the linkage control unit 211 transmits shared information to the organization 110 of the linkage destination based on the personal scenario (Step S204). Specifically, the following processes are executed.

(S204-1) The linkage control unit 211 refers to an entry corresponding to the personal scenario to search for a row (linkage information) in which the type of the received trigger information is set to the trigger information 613. The linkage control unit 211 acquires the type of service from the linkage destination 612 of the searched row (linkage information).

(S204-2) The linkage control unit 211 refers to the organization management information 220 to search for an entry in which the acquired type of service is stored in the service type 303. The linkage control unit 211 determines the organization 110 corresponding to the searched entry as the linkage destination.

(S204-3) The linkage control unit 211 generates shared information from the trigger information based on the item 614 of the searched row (linkage information).

For example, the linkage control unit 211 may generate the trigger information as the shared information as it is, or may generate the shared information in which a part of the trigger information is masked. The linkage control unit 211 may perform data format conversion or the like as needed. In addition, when plural organizations 110 of the linkage destination exist, the linkage control unit 211 may generate the shared information that differs depending on each organization 110.

(S204-4) The linkage control unit 211 transmits the generated shared information to the determined organization 110.

Note that before transmitting the shared information, the linkage control unit 211 may make an inquiry including personal information or the like included in the shared information to identify the organization 110 related to the user 101. The above is the description of the process of Step S204.

Next, the linkage control unit 211 updates the implementation scenario management information 224 (Step S205), and then terminates the linkage control process.

Specifically, the linkage control unit 211 sets information regarding the service of the organization 110 of the linkage destination 612 to the transition state 704 of the entry added to the implementation scenario management information 224 in Step S203.

When it is determined that the linkage control is continued in Step S202, the linkage control unit 211 transmits the shared information to the organization 110 of the linkage destination based on the personal scenario (Step S206). The process of Step S206 is the same as that of Step S204.

Next, the linkage control unit 211 updates the implementation scenario management information 224 (Step S207). The process of Step S207 is the same as that of Step S205.

Next, the linkage control unit 211 determines whether or not to terminate the linkage control (Step S208). That is, it is determined whether or not the shared information has been transmitted to all the organizations 110 of the linkage destination set to the personal scenario.

Specifically, the linkage control unit 211 determines whether or not all the types of trigger information set to the personal scenario are stored in the trigger information 703 of the entry of the implementation scenario management information 224. If all the types of trigger information set to the personal scenario are stored in the trigger information 703 of the entry of the implementation scenario management information 224, the linkage control unit 211 determines that the linkage control is terminated.

If it is determined that the linkage control is continued, the linkage control unit 211 terminates the linkage control process.

If it is determined that the linkage control is terminated, the linkage control unit 211 deletes the entry from the implementation scenario management information 224 (Step S209), and then terminates the linkage control process.

As described above, according to the first embodiment, the linkage of information between the organizations can be realized without performing an explicit operation. That is, plural services can be automatically provided by propagating information between the organizations 110 with the occurrence of the event related to the user 101. Necessary or useful services can be automatically and quickly provided to the user 101 and the person involved 102 without voluntary declaration of intention of the user 101.

### Second Embodiment

In a second embodiment, the configuration for realizing the linkage infrastructure system 100 differs. Hereinafter, the second embodiment will be described by mainly focusing on the difference from the first embodiment.

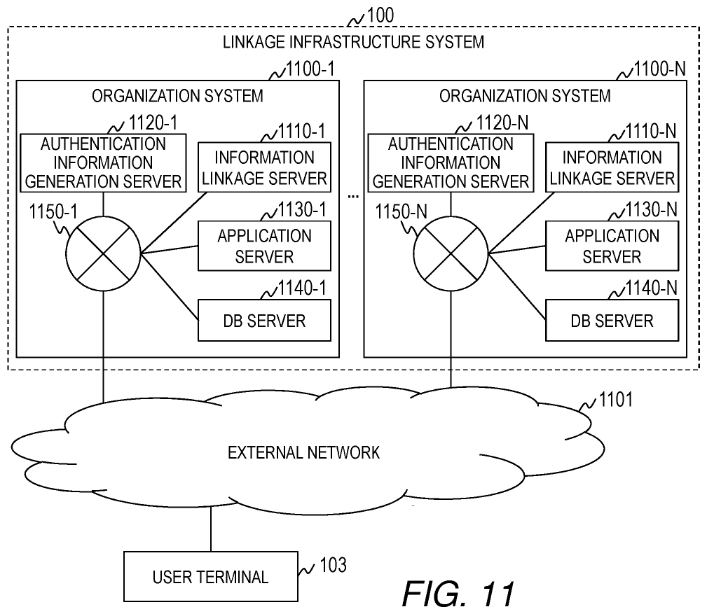

FIG. 11 is a diagram for showing a configuration example of a linkage infrastructure system 100 of the second embodiment.

In the second embodiment, the linkage infrastructure system 100 consists of systems (organization systems) 1100 operated by an organization 110. In FIG. 11, the linkage infrastructure system 100 consists of N organization systems 1100.

The respective organization systems 1100 are connected to each other through an external network 1101. Each organization system 1100 includes an information linkage server 1110, an authentication information generation server 1120, an application server 1130, and a database server 1140. The respective servers are connected to each other through an internal network 1150.

The external network 1101 is a communication network that connects between the organizations 110 and between the organizations 110 and the user 101. The internal network 1150 is a communication network that connects the respective servers in the organization system 1100 to each other. The external network 1101 and the internal network 1150 are, for example, the Internet, an intranet, a WAN, a LAN, and the like. The connection method may be either wired or wireless.

Note that the organization systems 1100 may be either on-premise systems or cloud systems.

Note that each server may consist of a physical computer or a virtual computer. In addition, each server may consist of plural physical computers or virtual computers for execution of distribution. Further, some or all of the servers may consist of one physical computer or virtual computer.

The information linkage server 1110 has the same function as the computer 200 of the first embodiment. The configuration of the information linkage server 1110 is the same as that of the computer 200, and thus the description thereof is omitted.

The application server 1130 executes an arbitrary process based on an execution request. For example, the application server 1130 executes a process for providing services in response to reception of shared information. The authentication information generation server 1120 generates authentication information used for identity authentication, and transmits the same to the information linkage server 1110. The authentication information may be any information such as a password and a public key, and is selected in accordance with the authentication intensity. The database server 1140 stores data managed by its own organization 110. For example, the personal information of the user 101, the contract contents of services, and the like are stored.

In the second embodiment, the user 101 transmits a registration request for a personal scenario to any of the organizations 110. The information linkage server 1110 of the organization system 1100 receiving the request executes a personal scenario registration process.

The personal scenario registration process of the second embodiment is different from the process of Step S104. Specifically, the information linkage server 1110 updates the personal scenario management information 223, and then transmits an update request including update contents to the organization system 1100 of another organization 110. When receiving the update request, the information linkage server 1110 included in the organization system 1100 of the other organization 110 updates the personal scenario management information 223 based on the update contents.

In the second embodiment, the information linkage server 1110 starts a linkage control process when the organization system 1100 detects newly-registered information and newly-generated information.

In the linkage control process of the second embodiment, the processes of Step S203, Step S206, Step S208, and Step S210 are partially different. Specifically, the information linkage server 1110 updates the implementation scenario management information 224, and then transmits an update request including update contents to the organization system 1100 of the other organization 110. When receiving the update request, the information linkage server 1110 included in the organization system 1100 of the other organization 110 updates the implementation scenario management information 224 based on the update contents.

The linkage infrastructure system 100 of the second embodiment has the same effect as the first embodiment.

### Third Embodiment

In a third embodiment, concrete use cases of the linkage infrastructure system 100 will be described.

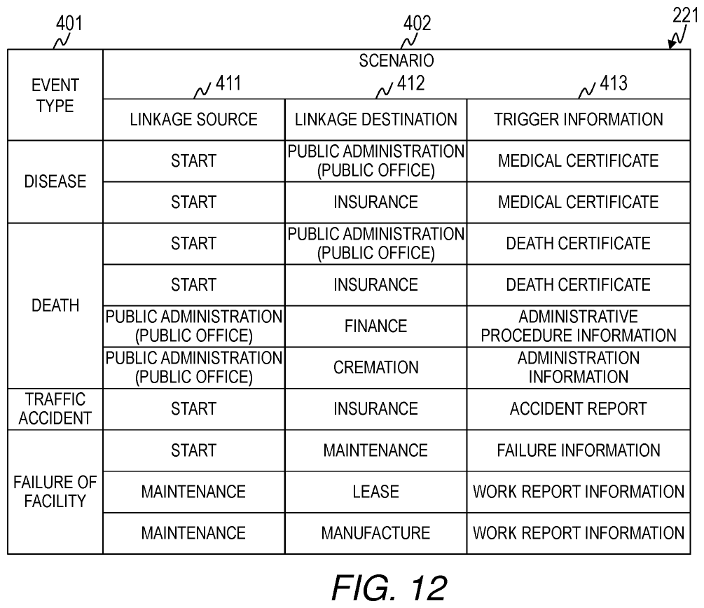

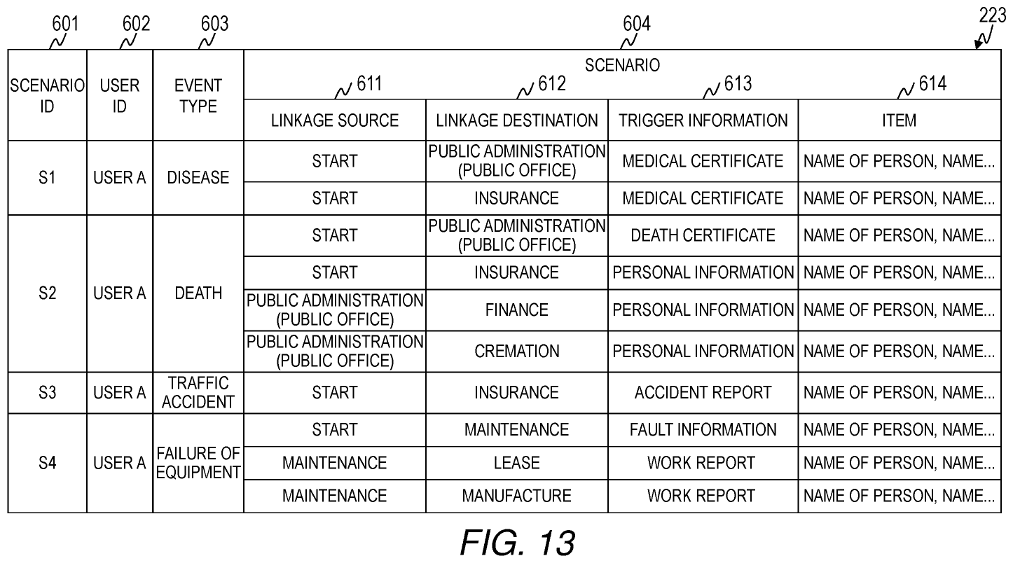

FIG. 12 is a diagram for showing a concrete example of the scenario template management information 221 of the third embodiment. FIG. 13 is a diagram for showing a concrete example of the personal scenario management information 223 of the third embodiment.

(1) Linkage Control Process Accompanying Disease of User A

First, a linkage control process accompanying a disease of a user A will be described. As shown in FIG. 12, trigger information for starting the linkage control is a medical certificate prepared by a doctor who works in a hospital. Here, it is assumed that a personal scenario (scenario ID 601 is “S1”) as shown in FIG. 13 has been set.

A system operated by the hospital transmits the medical certificate of the user A to the linkage infrastructure system 100.

Since the personal scenario of the user A exists (YES in Step S201) and the received trigger information is the trigger information corresponding to the start of the linkage control (YES in Step S202), the linkage control unit 211 transmits the medical certificate to a public office and an insurance company (Step S204).

Note that if a specific public office and insurance company are not designated, the linkage control unit 211 transmits the medical certificate to all the public offices and insurance companies connected to the linkage infrastructure system 100.

When receiving the medical certificate, the public office determines whether or not the user A resides based on the personal information included in the medical certificate. When the residence of the user A can be confirmed, the public office performs various administrative procedures. For example, an application form and the like are prepared.

When receiving the medical certificate, the insurance company determines whether or not the contract of the user A exists based on the personal information included in the medical certificate. When the contract of the user A can be confirmed, the insurance company determines the payment amount, and deposits the payment amount to a predetermined account.

As described above, the disease of the user 101 can be automatically detected, and information can be linked between the organizations 110. In addition, automatic administrative procedures, automatic payment of an insurance claim, and the like can be realized.

(2) Linkage Control Process Accompanying Death of User A

Next, a linkage control process accompanying the death of the user A will be described. As shown in FIG. 12, trigger information for starting the linkage control is a death certificate prepared by a doctor who works in a hospital. Here, it is assumed that a personal scenario (scenario ID 601 is “S2”) as shown in FIG. 13 has been set.

A system operated by the hospital transmits the death certificate of the user A to the linkage infrastructure system 100.

Since the personal scenario of the user A exists (YES in Step S201) and the received trigger information is the trigger information corresponding to the start of the linkage control (YES in Step S202), the linkage control unit 211 transmits the death certificate to a public office and an insurance company (Step S204).

Note that if a specific public office and insurance company are not designated, the linkage control unit 211 transmits the death certificate to all the public offices and insurance companies connected to the linkage infrastructure system 100.

When receiving the death certificate, the public office determines whether or not the user A resides based on the personal information included in the death certificate. When the residence of the user A can be confirmed, the public office performs various administrative procedures. For example, preparation and acceptance of a corpse cremation and burial certificate, procedures for receiving a pension, procedures for losing a nursing-care insurance qualification, procedures for canceling a residence certificate, and procedures for changing a householder are carried out. The public office transmits administrative procedure information including the personal information and the result of the administrative procedures to the linkage infrastructure system 100.

When receiving the death certificate, the insurance company determines whether or not the contract of the user A exists based on the personal information included in the death certificate. When the contract of the user A can be confirmed, the insurance company determines the payment amount, and deposits the payment amount to a predetermined account (for example, the account of the person involved 102).

When receiving the administrative procedure information from the public office, the linkage control unit 211 transmits the administrative procedure information to a financial institution and a crematorium because the personal scenario of the user A exists (YES in Step 5201) and the linkage control is continued (NO in Step S202) (Step S206).

Note that if a specific financial institution and crematorium are not designated, the linkage control unit 211 identifies the neighboring financial institution and crematorium based on the location of the public office and the like, and transmits the administrative procedure information to the identified financial institution and crematorium.

When receiving the administrative procedure information, the financial institution determines whether or not the account of the user A exists based on the personal information included in the administrative procedure information. When the existence of the account of the user A can be confirmed, the financial institution performs procedures such as freezing of the account, and notifies the person involved 102 of asset information and a method of canceling the freezing of the account.

When receiving the administrative procedure information, the crematorium notifies the person involved 102 of the available time of acceptance. The person involved 102 can make an inquiry to a funeral company based on the available time of acceptance. In this case, the funeral company can present schedules of a wake, a funeral, and the like to the person involved 102 based on the time.

As described above, the death of the user 101 can be automatically detected, and information can be linked between the organizations 110. In addition, automatic administrative procedures, automatic payment of an insurance claim, automatic inquiries, and the like can be realized. Even if there is no voluntary action of the user, various services can be provided based on the prior intention of the user 101.

(3) Linkage Control Process Accompanying Traffic Accident of User A

Next, a linkage control process accompanying a traffic accident of the user A will be described. As shown in FIG. 12, trigger information for starting the linkage control is an accident report prepared by a police officer. Here, it is assumed that a personal scenario (scenario ID 601 is “S3”) as shown in FIG. 13 has been set.

The user A reports the occurrence of the traffic accident to a police station. A system operated by the police station transmits the accident report prepared by the police officer to the linkage infrastructure system 100. The accident report includes the personal information of the user A such as a license number.

Note that the accident report includes personal information of a person involved in the traffic accident such as a license number or insurer number in some cases.

Since the personal scenario of the user A exists (YES in Step S201) and the received trigger information is the trigger information corresponding to the start of the linkage control (YES in Step S202), the linkage control unit 211 transmits the accident report to an insurance company (Step S204).

Note that if a specific insurance company is not designated, the linkage control unit 211 transmits the accident report to all the insurance companies connected to the linkage infrastructure system 100.

When receiving the accident report, the insurance company determines whether or not the contract of the user A exists based on the personal information included in the accident report. When the contract of the user A can be confirmed, the insurance company determines the payment amount, and deposits the payment amount to a predetermined account.

When the personal information of a person involved is included in the accident report, the insurance company determines whether or not the contract of the user A or person involved exists based on the personal information included in the accident report. When the contract of the user A or person involved can be confirmed, the insurance company consults with another insurance company, and determines the payment amount to deposit the payment amount to the predetermined account of the user A or person involved.

As described above, the traffic accident of the user 101 can be automatically detected, and information can be linked between the organizations 110. In addition, a consultation between the insurance companies, payment of an insurance claim, and the like can be realized. Even if there is no voluntary action of the user, various services can be provided based on the prior intention of the user 101.

(4) Linkage Control Process Accompanying Failure of Equipment

Next, a linkage control process accompanying a failure of leased equipment of the user A will be described. As shown in FIG. 12, trigger information for starting the linkage control is failure information prepared by an operator of a reception center. Here, it is assumed that a personal scenario (scenario ID 601 is “S4”) as shown in FIG. 13 has been set.

The user A reports the failure of the equipment to the reception center. A system operated by the reception center transmits the failure information prepared by the operator to the linkage infrastructure system 100. The failure information includes information of the company to which the user A belongs or personal information of the user A.

Since the personal scenario of the user A exists (YES in Step S201) and the received trigger information is the trigger information corresponding to the start of the linkage control (YES in Step S202), the linkage control unit 211 transmits fault information to a maintenance company (Step S204).

Note that if a specific maintenance company is not designated, the linkage control unit 211 transmits the fault information to all the maintenance companies connected to the linkage infrastructure system 100.

When receiving the fault information, the maintenance company determines the schedule of maintenance work with the user A based on the personal information included in the accident report. In addition, a worker of the maintenance work prepares a work report including the contents of the maintenance work and the like. A system operated by the maintenance company transmits work information to the linkage infrastructure system 100. The work information includes personal information of the user A and information of the equipment.

When receiving the work information, the linkage control unit 211 transmits the work report to a leasing company and a manufacturing company because the personal scenario of the user A exists (YES in Step 5201) and the received trigger information indicates that the linkage control is continued (NO in Step 5202) (Step S206).

Note that if a specific leasing company is not designated, the linkage control unit 211 makes an inquiry including the personal information of the user A included in the work report to all the leasing companies connected to the linkage infrastructure system 100. Accordingly, the work report can be transmitted to the leasing company having a contract with the user A.

Note that if a specific manufacturing company is not designated, the linkage control unit 211 makes an inquiry including the information of the equipment included in the work report to all the manufacturing companies connected to the linkage infrastructure system 100. Accordingly, the work report can be transmitted to the manufacturing company that manufactures the equipment.

As described above, the failure of the equipment used by the user 101 can be automatically detected, and information can be linked between the organizations 110. In addition, even if the user 101 and the maintenance company are changed, the operation quality of the equipment is improved by continuously providing the maintenance service corresponding to the equipment. In addition, the operation cost of the leasing company is improved.

Note that the present invention is not limited to the above-described embodiments, and includes various modified examples. In addition, for example, the configurations of the embodiments have been described in detail to easily understand the present invention, and the present invention is not necessarily limited to those including all the configurations described above. In addition, some configurations of each embodiment can be added to, deleted from, and replaced by other configurations.

In addition, the above-described configurations, functions, processing units, processing means, and the like may be realized using hardware by, for example, designing some or all thereof with an integrated circuit. In addition, the present invention can be also realized by a program code of software that realizes the functions of the embodiments. In this case, a storage medium in which a program code is recorded is provided to a computer, and a processor provided in the computer reads the program code stored in the storage medium. In this case, the program code itself read from the storage medium realizes the functions of the above-described embodiments, and the program code itself and the storage medium storing the program code configure the present invention. As a storage medium for supplying such a program code, for example, a flexible disk, a CD-ROM, a DVD-ROM, a hard disk, an SSD (Solid State Drive), an optical disk, a magneto-optical disk, a CD-R, a magnetic tape, a nonvolatile memory card, a ROM, or the like is used.

In addition, the program code for realizing the functions described in the embodiments can be implemented using a wide range of programs or scripting languages such as, for example, an assembler, C/C++, perl, Shell, PHP, Python, Java (registered trademark), and the like.

Further, a program code of software that realizes the functions of the embodiments may be distributed through a network to be stored in storage means such as a hard disk or a memory of a computer or a storage medium such as a CD-RW or a CD-R, and a processor provided in the computer may read and execute the program code stored in the storage means or the storage medium.

In addition, the control lines and the information lines considered to be necessary in the explanation are shown in the above-described embodiments, and all the control lines and the information lines in a product are not necessarily shown. All the configurations may be connected to each other.

1. A computer system for performing linkage control of information between organizations, the system comprising at least one computer having an arithmetic device and a storage device connected to the arithmetic device,
wherein scenario management information for managing a scenario in which trigger information related to an event related to a user is associated with a target organization as a target of the linkage control accompanying an occurrence of the event is held, and
wherein when receiving information from an arbitrary organization, the arithmetic device refers to the scenario management information to search for the scenario including the received information as the trigger information, identifies the target organization based on the scenario and the received information, and transmits shared information generated from the received information to the target organization.

2. The computer system according to claim 1,
wherein the scenario includes identification information of the user, a type of event, and at least one linkage information including the trigger information, the target organization, and item information defining items to be included in the shared information, and
wherein the arithmetic device refers to the scenario management information to search for the scenario including the identification information of the user included in the received information and the linkage information including the received information, generates the shared information from the received information based on the item information included in the linkage information including the received information of the searched scenario, and transmits the shared information to the target organization included in the linkage information including the received information of the searched scenario.

3. The computer system according to claim 2,
wherein scenario template management information storing first data including the type of event, the trigger information, and the target organization is held, and
wherein the arithmetic device presents a screen for setting the scenario based on the scenario template management information, accepts registration information including the first data, the identification information of the user, and the item information through the screen, generates the scenario based on the registration information, and registers the same in the scenario management information.

4. The computer system according to claim 3,
wherein the scenario template management information stores:
the first data including a disease of the user, a medical certificate, and an organization providing administrative services; and
the first data including the disease of the user, information generated by the organization providing the administrative services, and an organization providing insurance services.

5. The computer system according to claim 3,
wherein the scenario template management information stores:
the first data including the death of the user, a death certificate, and an organization providing administrative services;
the first data including the death of the user, the death certificate, and an organization providing insurance services;
the first data including the death of the user, information generated by the organization providing administrative services, and an organization providing financial services; and
the first data including the death of the user, information generated by the organization providing administrative services, and an organization providing services related to a funeral.

6. The computer system according to claim 3,
wherein the scenario template management information stores the first data including a traffic accident, an accident report, and an organization providing insurance services.

7. The computer system according to claim 3,
wherein the scenario template management information stores:
the first data including a failure of equipment used by the user, failure information, and an organization providing maintenance services for the equipment;
the first data including the failure of the equipment, information generated by the organization providing maintenance services for the equipment, and an organization providing lease services for the equipment; and
the first data including the failure of the equipment, the information generated by the organization providing maintenance services for the equipment, and an organization manufacturing the equipment.

8. A linkage control method of information between organizations executed by a computer system,
wherein the computer system includes at least one computer having an arithmetic device and a storage device connected to the arithmetic device,
wherein scenario management information for managing a scenario in which trigger information related to an event related to a user is associated with a target organization as a target of the linkage control accompanying an occurrence of the event is held, and
wherein the linkage control method comprises:
a first step of, when receiving information from an arbitrary organization, referring to the scenario management information to search for the scenario including the received information as the trigger information by the arithmetic device;
a second step of identifying the target organization based on the scenario and the received information by the arithmetic device; and
a third step of transmitting shared information generated from the received information to the target organization by the arithmetic device.

9. The linkage control method according to claim 8,
wherein the scenario includes identification information of the user, a type of event; and at least one linkage information including the trigger information, the target organization, and item information defining items to be included in the shared information,
wherein the first step includes a step of referring to the scenario management information to search for the scenario including the identification information of the user included in the received information and the linkage information including the received information by the arithmetic device, and
wherein the third step includes:
a step of generating the shared information from the received information based on the item information included in the linkage information including the received information of the searched scenario by the arithmetic device; and
a step of transmitting the shared information to the target organization included in the linkage information including the received information of the searched scenario by the arithmetic device.

10. The linkage control method according to claim 9,
wherein the computer system holds scenario template management information storing first data including the type of event, the trigger information, and the target organization, and
wherein the linkage control method comprises:
a step of presenting a screen for setting the scenario based on the scenario template management information by the arithmetic device;
a step of accepting registration information including the first data, the identification information of the user, and the item information through the screen by the arithmetic device; and
a step of generating the scenario based on the registration information and registering the same in the scenario management information by the arithmetic device.

11. The linkage control method according to claim 10,
wherein the scenario template management information stores:
the first data including a disease of the user, a medical certificate, and an organization providing administrative services; and
the first data including the disease of the user, information generated by the organization providing the administrative services, and an organization providing insurance services.

12. The linkage control method according to claim 10,
wherein the scenario template management information stores:
the first data including the death of the user, a death certificate, and an organization providing administrative services;
the first data including the death of the user, the death certificate, and an organization providing insurance services;
the first data including the death of the user, information generated by the organization providing administrative services, and an organization providing financial services; and
the first data including the death of the user, information generated by the organization providing administrative services, and an organization providing services related to a funeral.

13. The linkage control method according to claim 10,
wherein the scenario template management information stores the first data including a traffic accident, an accident report, and an organization providing insurance services.

14. The linkage control method according to claim 10,
wherein the scenario template management information stores:
the first data including a failure of equipment used by the user, failure information, and an organization providing maintenance services for the equipment;
the first data including the failure of the equipment, information generated by the organization providing maintenance services for the equipment, and an organization providing lease services for the equipment; and
the first data including the failure of the equipment, the information generated by the organization providing maintenance services for the equipment, and an organization manufacturing the equipment.
